(19) United States
(12) Patent Application Publication
(10) Pub. No.: US 2024/0307042 A1
(43) Pub. Date: Sep. 19, 2024

(54) INSTRUMENT FOR TAKING A TISSUE SAMPLE

(71) Applicant: Jacques Phillibert Janssens, Hasselt (BE)

(72) Inventor: Jacques Phillibert Janssens, Hasselt (BE)

(21) Appl. No.: 18/541,967

(22) Filed: Dec. 15, 2023

(30) Foreign Application Priority Data
Mar. 13, 2023 (BE) 2023/5182

(51) Int. Cl. A61B 10/02

(52) U.S. Cl. CPC A61B 10/0275

(57) ABSTRACT

An instrument for taking a tissue sample is provided with a tissue receiving element that has a distal end and partly consists of a helix comprising the distal end and has an outer surface, an inner surface, and a central longitudinal axis. The helix has a zone whereby for each point of a first intersecting line of the outer surface and a plane of which the central longitudinal axis forms part the distance from the outer surface to the longitudinal axis is less than the distance between the outer surface and the longitudinal axis on every more proximally located point, and whereby for each point of a second intersecting line of the inner surface and an aforementioned plane, the distance from the inner surface to the longitudinal axis is greater than the distance from the inner surface to the longitudinal axis on every more proximally located point.

### CROSS-REFERENCE TO RELATED APPLICATIONS

This application claims priority to Belgium Patent Application No. 2023/5182 filed on Mar. 13, 2023, which is incorporated herein by reference in its entirety.

### TECHNICAL FIELD

The present disclosure relates to an instrument for taking a tissue sample.

### BACKGROUND

More specifically it relates to an instrument of the type described in WO 02/065919, or in BE 1028018 A1, in which a spiral or helical tissue receiving element is turned in the tissue from which a sample must be taken, after which the tissue around this tissue receiving element is cut with a sharp cannula around the tissue receiving element and is torn off at the distal end of the tissue receiving element, thereby obtaining a tissue sample in the tissue receiving element.

Such instruments have a greater tissue sample volume/needle diameter ratio than a so-called tru-cut biopsy needle.

EP 2623036 B1 discloses an improvement of the aforementioned instrument, whereby the diameter of the outer surface of the spiral or helical tissue receiving element decreases toward the distal end of the tissue receiving element.

Consequently a space is created between the cutting cannula and the tissue receiving element when the cannula is slid over the tissue receiving element.

This has the aspect that when the spiral or helical tissue receiving element were to expand when being turned into harder tissues, the desired movement of the cutting cannula is not disrupted.

In addition, the aforementioned space also offers room for the so-called lateral connective tissue fibres that run through the tissue of organs and extend partly transversely to the longitudinal direction of the tissue receiving element and that are not cut on the outside of the helix.

Consequently, the tissue sample is held between the helix bodies such that the distal tearing will not cause tissue loss.

Most such instruments for taking a tissue sample have a small diameter.

For endoscopic applications a scope with a working channel is used in which the instrument for taking a tissue sample is inserted.

The diameter of the working channel is linked to the distance over which the endoscope can be inserted in the patient. Thus, the endoscope can be inserted maximally until the diameter of the organ, for example the airway, becomes smaller than the diameter of the scope.

The most common endoscopes have a working channel with a diameter that can only amount to 1.8 millimetres.

The result is that the diameter of the outer surface of the spiral or helical tissue receiving element can maximally only amount to less than 3 millimetres.

The result is that the tissue sample is very small.

Preclinical tests have shown that the dimensions of the tissue sample decrease over proportionally as the diameter of the tissue receiving element gets smaller.

However, in order to obtain a good and representative tissue sample, care must be taken to collect as much tissue as possible.

US 2012/0197157 A1 discloses an instrument for taking a biopsy with a rotatable coil that protrudes from a hollow needle.

US 2020/0015852 A1 discloses a needle for taking a biopsy that is provided with an expandable element to protect the sharp tip of the needle.

Both the disclosed biopsy instrument and the needle are unsuitable for generating a greater and solid tissue sample volume.

The purpose of the present disclosure is to provide a solution to at least one of said and other disadvantages by providing a device which allows the size of the tissue sample to be increased for one and the same diameter of the tissue receiving element.

### SUMMARY

To this end, the present disclosure relates to an instrument for taking a tissue sample, said instrument comprising a tissue receiving element with a distal end and a proximal end and at least partly consisting of a spiral or helix, said spiral or helix comprising the distal end and having an outer surface and an inner surface and a central longitudinal axis, whereby the spiral or helix at least has a first zone whereby for each point of a first intersecting line of the outer surface and a plane of which the central longitudinal axis forms part, the distance from the outer surface to the central longitudinal axis is less than or equal to the distance from the outer surface to the central longitudinal axis on every more proximally located point along said first intersecting line, whereby the spiral or helix at least has a second zone whereby for each point of a second intersecting line of the inner surface and a plane of which the central longitudinal axis forms part, the distance from the inner surface to the central longitudinal axis (X-X′) is greater than or equal to the distance from the inner surface to the central longitudinal axis on every more proximally located point along said second intersecting line.

In some embodiments, the first and the second zone fully coincide and comprise the distal end of the tissue receiving element.

“Comprising the distal end” is understood to mean here that the distal end is part of the spiral or helix, or of the first or second zone.

In some embodiments, the spiral or helix of the tissue receiving element will taper off toward the distal end at its outer surface, and get wider toward the distal end at its inner surface. This tapering and widening at the outer surface and the inner surface may occur independently from each other.

This provides the aspect that the internal volume of the tissue receiving element is greater compared to the known instruments, such that a greater tissue sample volume can be taken.

Moreover, as a result of this the thickness of the spiral or helix, i.e. the distance between the inner surface and the outer surface, will decrease toward the distal end.

Another aspect is that the section of the spiral or helix at the distal end will be more flexible.

When the tissue receiving element is turned in the tissue, the distal end, or the tip of the spiral or helix, upon penetration of the spiral or helix, will tend to expand, such that a greater circle is described by the tip.

While turning the tissue receiving element, the spiral or helix will thus expand. In other words: the volume of the tissue sample will be greater than the volume enclosed by the spiral or helix before it is turned in the tissue.

Another aspect is that the spiral or helix will be thicker, and therefore less flexible at the proximal end, such that the spiral or helix has a greater stability.

It is exactly said zone at the proximal end that is exposed to the greatest torsional forces during use.

In a practical embodiment the instrument also comprises a tubular cutting element that has a distal end with a cutting edge and that fits around the tissue receiving element.

Said cutting edge can assume different forms, such as flat, tooth-shaped, etc.

In some embodiments, the tubular cutting element will cut loose the tissue sample from the surrounding tissue by sliding the cutting element to the distal end and turning it over the tissue receiving element clockwise.

The flexibility of the spiral or helix will facilitate sliding the tubular cutting element over the tissue receiving element, even when said spiral is expanded.

Consequently the tissue is lightly compressed in the tissue receiving element, such that said tissue is fixed better in the tissue receiving element. This prevents the risk of loss of tissue upon withdrawing the instrument from the tissue.

According to an embodiment of the present disclosure the inner surface and the outer surface of the spiral or helix are connected to each other by means of lateral surfaces, whereby the intersecting lines of the lateral surfaces with a plane of which the central longitudinal axis forms part, enclose an angle with the central longitudinal axis that is less than 90°, said angle pointing in proximal direction.

As a result of this, said lateral surfaces will be at an angle with the central longitudinal axis, such that a kind of barb effect is created, whereby the lateral surfaces becomes barbs as it were.

Consequently, the cross-section of the spiral or helix with a plane of which the central longitudinal axis forms part, will not be rectangular, but a parallelogram or trapezoid with an acute angle in proximal direction.

This barb effect will ensure that the tissue sample is fixedly hooked when cutting and when withdrawing the instrument from the tissue.

This hooking effect will be greater the more said angle deviates from 90°.

As a result of this, the cut off tissue sample will slide off the tissue receiving element less easily when cutting with the tubular cutting element and withdrawing the tissue receiving element from the tissue.

### BRIEF DESCRIPTION OF THE DRAWINGS

With the intention of better showing the characteristics of the present disclosure a few embodiments of an instrument for taking a tissue sample according to the present disclosure are described hereinafter, by way of an example without any limiting nature, with reference to the accompanying drawings, wherein:

FIG. 1 schematically shows a perspective view of an instrument for taking a tissue sample according to the present disclosure;

FIG. 2 schematically shows the tissue receiving element of FIG. 1;

FIG. 3 shows a cross-section of FIG. 2;

FIG. 4 shows the section indicated in FIG. 3 with F4 on a larger scale.

### DETAILED DESCRIPTION

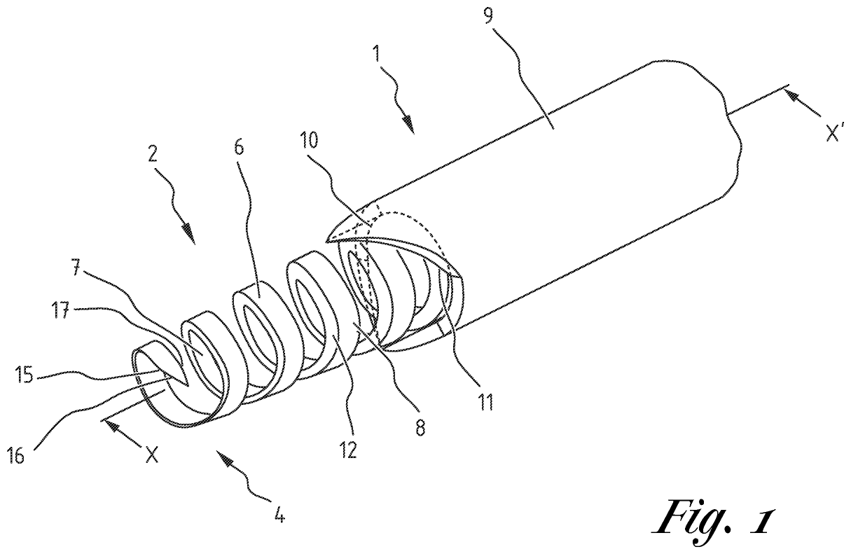

The instrument 1 for taking a tissue sample schematically shown in FIG. 1 comprises a tissue receiving element 2.

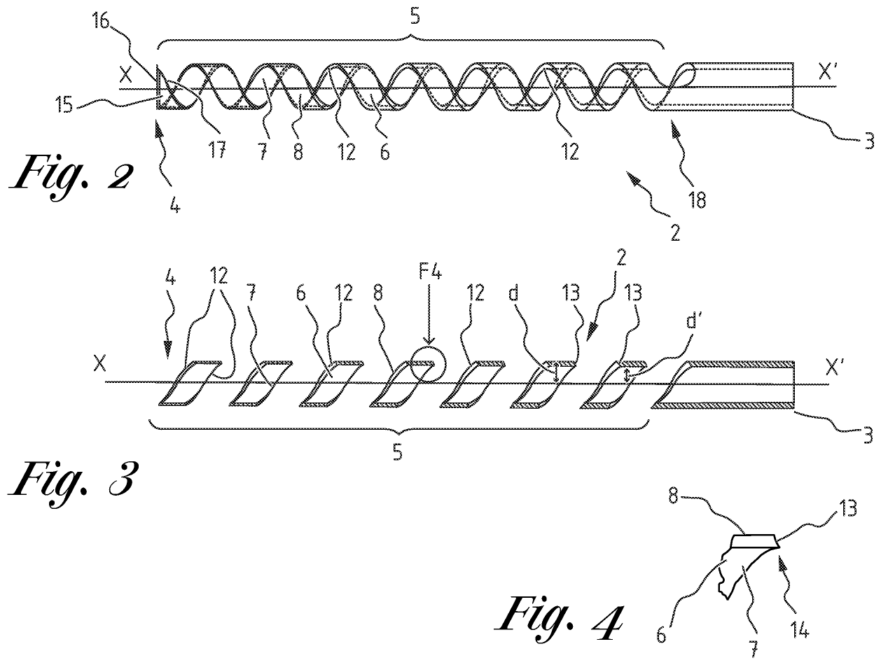

The tissue receiving element 2 is shown in detail in FIG. 2.

The tissue receiving element 2 has a proximal end 3 and a distal end 4.

A section of the tissue receiving element 2 is formed as a spiral or helix 6. The spiral or helix 6 is situated at the distal end 4, whereas the proximal end 3 is tubular.

The spiral or helix 6 comprises an inner surface 7 and an outer surface 8 and a central longitudinal axis X-X′.

The central longitudinal axis X-X′ of the helix or spiral 6 coincides with the central longitudinal axis X-X′ of the proximal tubular proximal end 3 of the tissue receiving element 2.

The spiral or helix 6 comprises a first zone 5 in which the spiral or helix 6 conically tapers off toward the distal end 4 at its outer surface 8.

Or in other words: at least for said first zone 5 of the spiral or helix 6, for each point of a first intersecting line of the outer surface 8 and a plane of which the central longitudinal axis (X-X′) forms part, the distance d from the outer surface 8 to the central longitudinal axis X-X′ will be less than or equal to the distance d from the outer surface 8 to the central longitudinal axis X-X′ on every more proximally located point along said first intersecting line.

According to the present disclosure, the spiral or helix 6 will comprise a second zone 5 in which the spiral or helix 6 conically widens toward the distal end 4 at its inner surface 7.

Or in other words: at least for said first zone 5 of the spiral or helix 6, for each point of a second intersecting line of the outer surface 7 and a plane of which the central longitudinal axis (X-X′) forms part, the distance d′ from the inner surface 7 to the central longitudinal axis X-X′ will be greater or equal to the distance d′ from the inner surface 7 to the central longitudinal axis X-X′ on every more proximally located point along said second intersecting line.

In the example of FIG. 2 the first zone 5 and the second zone 5 fully coincide.

Moreover, the first zone 5 and the second zone 5 comprise the distal end 4.

A result of this is that the thickness of the spiral or helix 6, or in other words the distance d-d′ between the inner surface 7 and the outer surface 9, can gradually decrease toward the distal end 4 of the spiral or helix 6.

The cross-section in FIG. 3 clearly shows the decrease of the thickness of the spiral or helix 6 toward the distal end 4.

Said decrease of the thickness ensures a certain flexibility of the spiral or helix 6, resulting in the aforementioned advantages.

Particularly the fact that as a result of this, the spiral or helix 6 will expand when turning into the tissue receiving element 2. In other words: the volume of the tissue sample will be greater than the volume enclosed by the spiral or helix 6 before it is turned into the tissue.

Furthermore, due to the internal conical widening of the spiral or helix 6 toward the distal end 4, the internal volume of the spiral or helix 6, i.e. the tubular/conical volume enclosing the spiral or helix 6, will be greater than in the known instruments for taking a tissue sample, for example tru-cut.

Next to the tissue receiving element 2, the instrument, according to the present disclosure, also comprises in this case a tubular cutting element 9 that fits around the tissue receiving element 2.

Or in other words: the tissue receiving element 2 is fittingly enclosed in the tubular cutting element 9.

FIG. 1 schematically shows an example of the cutting element 9.

In this case the cutting element 9 will fit closely rotatively and slideably lengthways around the tissue receiving element 2.

The cutting element 9 has a cutting edge 11 at its distal end 10.

The shape and size of the cutting element 9 and the tissue receiving element 2 are such that when used they cooperatively exert a cutting effect on tissue surrounding the spiral or helix 6.

The flexibility of the spiral or helix 6 will facilitate sliding the tubular cutting element 9 over the tissue receiving element 2, even when said spiral is expanded, resulting in a slight compression of the tissue sample. The resilience of the distal end 4 of the spiral or helix 6 also ensures the necessary contact between the sharp outer edge of the spiral or helix 6 and the cutting element 9.

The cross-section in FIG. 3 shows that the inner surface 7 and the outer surface 8 of the spiral or helix are connected with each other by lateral surfaces 12.

The cross-section in FIG. 3 is made according to a plane of which the central longitudinal axis (X-X′) forms part.

The intersecting lines 13 of the lateral surfaces 12 with a plane of which the central longitudinal axis (X-X′) forms part, are therefore visible in FIG. 3.

Said intersecting lines 13 enclose an angle with the central longitudinal axis X-X′ that is less than 90°, said angle pointing in proximal direction.

FIG. 4 shows a detail, which shows that in this way a kind of barb 14 is created as it were.

However, said barb 14 does not extend outside the outer surface 8 of the spiral or helix 6, and thus cannot obstruct the tubular cutting element 9, nor does it extend past the inner surface 7 of the spiral or helix 6, and thus cannot damage the tissue sample.

The barb 14 will only engage on the intersecting line made in the tissue, to anchor in the tissue sample there.

The spiral or helix 6 is provided with a tip 15 at the distal end 4 of the tissue receiving element 2.

To this end, the distal end 4 of the spiral or helix 2 is cut off according to a plane perpendicular to the central longitudinal axis X-X′, such that the tip 15 is formed as a right-angled triangle, one of the right-angled sides 16 of which is formed by said cut off.

The thus created hypotenuse 17 of the tip 15, which is formed by the side of the spiral or helix 2 causes the tip 15 to lean over more towards the distal end when loading and turning the instrument 1 in the tissue, such that the helix 2 immediately hooks into the tissue. This facilitates the biopsy procedure in cavities

Due to the decrease of the thickness of the spiral or helix 6 toward the distal end 4, said tip will be very thin, which is useful for the production of the tissue receiving element 2.

Because there is little material at the distal end 4 of the helix or spiral 6, upon cutting off the tip 15 during production there will be little to no overheating, and as consequence of this no burr formation either.

Burr formation may not only make the tip 15 less sharp but also fragile, with a risk of breaking off when taking a biopsy.

This risk can thus be completely avoided here.

At the proximal end 18 of the spiral or helix 6, the spiral or helix 6 is attached or connected to the tubular proximal end 3 of the tissue receiving element 2.

In the transition from the spiral or helix 6 to the tubular proximal end 3, the spiral or helix 6 has a greater radius such that the attachment of the spiral or helix 6 on the tubular proximal end 3 of the tissue receiving element 2 is widened.

Due to this configuration the attachment of the spiral or helix 6 to the tubular proximal end 3 of the tissue receiving element 2 is stronger, such that the rotational or tensile force n on the helix 6 can become greater without the risk of tearing or breaking.

The operation of the instrument according to the present disclosure is very simple and as follows.

First a localisation needle or catheter, at least composed of a so-called trocar or wire and the cutting element 9, is inserted in the location where a tissue sample needs to be taken. The positioning is done using medical imaging either radiologically or endoscopically. Once in the right spot, the helix 6 can be positioned to be turned into the tissue.

To this end, the tissue receiving element 2 is inserted through the cutting element 9. A rotational movement now turns this tissue receiving element 2 in the tissue from which a sample must be taken. The tissue in the tissue receiving element 2 is not disturbed hereby, due to the fibres running from the space within the tissue receiving element 2 to the surroundings.

As described above, the spiral or helix 6 will slightly expand, such that a greater volume of tissue is enclosed by the tissue receiving element 2.

Subsequently the cutting element 9 is moved in a distal direction, while making a rotational motion simultaneously. Jointly thanks to the interaction between the tissue receiving element 2 and the cutting element 9, tissue around the tissue receiving element 2 is cut loose.

The expansion of the spiral or helix 6 causes the cutting element 9 to exert a slight compression on the spiral or helix 6, and consequently on the tissue sample.

Subsequently, a pulling power is exerted on the instrument 1 in a proximal direction, i.e. in the direction away from the tissue. Consequently, the tissue sample tears off near the distal end 4 of the tissue receiving element 2, and it can be brought outside the patient's body for the required analyses.

Said barb effect and slight compression that the cutting element 9 exerts is important here, such that a significant force can be exerted on the tissue sample via the tissue receiving element 2, resulting in the tissue sample tearing off, and with little risk of sample loss when the whole tissue receiving element 2 is withdrawn from the tissue.

The present disclosure is not limited to the embodiments described as an example and shown in the drawings, but an instrument for taking a tissue sample according to the present disclosure can be realised in all kinds of forms and dimensions without departing from the scope of the present disclosure.

What is claimed is:

1. An instrument for taking a tissue sample, the instrument comprising:
a tissue receiving element that has a distal end and a proximal end and at least partly formed as a spiral or helix, the spiral or helix comprising the distal end and having an outer surface and an inner surface and a central longitudinal axis, whereby the spiral or helix at least has a first zone whereby for each point of a first intersecting line of the outer surface and a plane of which the central longitudinal axis forms part, a distance from the outer surface to the central longitudinal axis is less than or equal to the distance from the outer surface to the central longitudinal axis on every more proximally point along the first intersecting line, and
a tubular cutting element fittingly applied around the tissue receiving element and at a distal end is provided with a cutting edge,
wherein the spiral or helix at least has a second zone whereby for each point of a second intersecting line of the inner surface and a plane of which the central longitudinal axis forms part, a distance from the inner surface to the central longitudinal axis is greater than or equal to the distance from the inner surface to the central longitudinal axis on every more proximally located point along the second intersecting line, and whereby the first and the second zone fully coincide and comprise the distal end of the spiral or helix.

2. The instrument according to claim 1, wherein the inner surface and the outer surface of the spiral or helix are connected to each other by lateral surfaces, whereby intersecting lines of the lateral surfaces with a plane of which the central longitudinal axis forms part, enclose an angle with the central longitudinal axis that is less than 90°, the angle pointing in proximal direction.

3. The instrument according to claim 1, wherein the distal end of the spiral or helix is cut off according to a plane perpendicular to the central longitudinal axis, such that the distal end of the spiral or helix is formed as a right-angled triangle of which one of a right-angled sides is formed by the cut off.

4. The instrument according to claim 1, wherein a most proximal end of the tissue receiving element is tubular to which the spiral or helix is connected, whereby in the transition from the spiral or helix to the tubular proximal end, the spiral or helix has a greater radius such that an attachment of the spiral or helix on the tubular proximal end of the tissue receiving element is widened.

5. The instrument according to claim 1, 1 wherein the tubular cutting element fits closely rotatively and slideably lengthways around the tissue receiving element.

6. The instrument according to claim 1, wherein a shape and size of the tubular cutting element and the tissue receiving element are such that when used they cooperatively exert a cutting effect on tissue surrounding the spiral or helix.

7. The instrument according to claim 2, wherein the distal end of the spiral or helix is cut off according to a plane perpendicular to the central longitudinal axis, such that the distal end of the spiral or helix is formed as a right-angled triangle of which one of a right-angled sides is formed by the cut off.

8. The instrument according to claim 2, wherein a most proximal end of the tissue receiving element is tubular to which the spiral or helix is connected, whereby in the transition from the spiral or helix to the tubular proximal end, the spiral or helix has a greater radius such that the attachment of the spiral or helix on the tubular proximal end of the tissue receiving element is widened.

9. The instrument according to claim 3, wherein a most proximal end of the tissue receiving element is tubular to which the spiral or helix is connected, whereby in the transition from the spiral or helix to the tubular proximal end, the spiral or helix has a greater radius such that the attachment of the spiral or helix on the tubular proximal end of the tissue receiving element is widened.

10. The instrument according to claim 7, wherein a most proximal end of the tissue receiving element is tubular to which the spiral or helix is connected, whereby in the transition from the spiral or helix to the tubular proximal end, the spiral or helix has a greater radius such that the attachment of the spiral or helix on the tubular proximal end of the tissue receiving element is widened.

11. The instrument according to claim 2, wherein a shape and size of the tubular cutting element and the tissue receiving element are such that when used they cooperatively exert a cutting effect on tissue surrounding the spiral or helix.

12. The instrument according to claim 3, wherein a shape and size of the tubular cutting element and the tissue receiving element are such that when used they cooperatively exert a cutting effect on tissue surrounding the spiral or helix.

13. The instrument according to claim 4, wherein a shape and size of the tubular cutting element and the tissue receiving element are such that when used they cooperatively exert a cutting effect on tissue surrounding the spiral or helix.

14. The instrument according to claim 5, wherein a shape and size of the tubular cutting element and the tissue receiving element are such that when used they cooperatively exert a cutting effect on tissue surrounding the spiral or helix.

15. The instrument according to claim 7, wherein a shape and size of the tubular cutting element and the tissue receiving element are such that when used they cooperatively exert a cutting effect on tissue surrounding the spiral or helix.

16. The instrument according to claim 8, wherein a shape and size of the tubular cutting element and the tissue receiving element are such that when used they cooperatively exert a cutting effect on tissue surrounding the spiral or helix.

16. The instrument according to claim 9, wherein a shape and size of the tubular cutting element and the tissue receiving element are such that when used they cooperatively exert a cutting effect on tissue surrounding the spiral or helix.

17. The instrument according to claim 10, wherein a shape and size of the tubular cutting element and the tissue receiving element are such that when used they cooperatively exert a cutting effect on tissue surrounding the spiral or helix.
